(19) United States
(12) Patent Application Publication
(10) Pub. No.: US 2006/0155283 A1
(43) Pub. Date: Jul. 13, 2006

(54) OCCIPITAL PLATE AND GUIDE SYSTEMS

(75) Inventors: Thomas V. Doherty, Bellingham, MA (US); Matthew Lake, Braintree, MA (US); Douglas LaSota, Saugus, MA (US); Michael Mazzuca, Bellingham, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE SARL, Le Locle (CH)

(21) Appl. No.: 10/905,503

(22) Filed: Jan. 7, 2005

(51) Int. Cl. A61F 2/30

(52) U.S. Cl. 606/69

(57) ABSTRACT

Disclosed herein are spinal fixation devices and tools for implanting the same. In one embodiment, an implantable spinal fixation plate and a guide device are provided and they include features that allow the two devices to removably mate to one another. As a result, the guide device can be used to position and hold the plate against bone while inserting drills, taps, awls, and other bone preparation devices through the guide device. The guide device can also be configured to allow fasteners to be inserted therethrough and into bone to attach the plate to bone.

### BACKGROUND

Treatment of some spinal injuries or disorders may involve the use of a spinal fixation element, such as a relatively rigid fixation rod, that is coupled to adjacent vertebrae by attaching the element to various anchoring devices, such as plates, hooks, bolts, wires, or screws. Often two rods are disposed on opposite sides of the spinous process in a substantially parallel relationship. The fixation rods can have a predetermined contour that has been designed according to the properties of the target implantation site, and once installed, the rods hold the vertebrae in a desired spatial relationship, until healing or spinal fusion has taken place, or for some longer period of time. When such surgery is performed in the cervical spine, the proximal ends of the rods are typically molded according to the anatomy of the skull and the cervical spine, and attached to a fixation plate that is implanted in the occiput.

While occipital bone plates provide a stable technique for occipito-cervical fixation, fixation to the occiput can be a challenge. In particular, each spinal plate must be properly aligned with the occiput, and holes for receiving the bone screws must be drilled into the occiput at precise angles. It is often necessary to use the spinal plate as a guide device for drilling and preparing the bone for receiving the bone screws. This can be difficult, however, as the surgeon is required to simultaneously hold the spinal plate against the occiput, obtain proper alignment, drill, tap, and finally implant the bone screws.

Accordingly, there remains a need for improved spinal fixation devices and tools for use in the spine, and in particular for improved methods and devices for implanting a spinal plate.

### SUMMARY

Disclosed herein are spinal fixation devices and tools for implanting the same. In one exemplary embodiment, a spinal fixation plate is provided having at least one thru-bore formed therein for receiving a fastener, such as a bone screw, for attaching the plate to bone. While the plate can have virtually any configuration, in one exemplary embodiment the plate is an occipital plate having an elongate central portion with proximal and distal ends that define a longitudinal axis extending therebetween. First and second branch portions can extend from opposed sides of the elongate central portion. The plate can also include at least one thru-bore formed in the elongate central portion of the plate and/or the branch portions. In an exemplary embodiment the elongate central portion includes two or three thru-bores formed therein along the longitudinal axis thereof for receiving a fastening element, and each branch portion includes a thru-bore or slot formed therein for receiving an anchoring element adapted to mate a spinal fixation element to the plate.

In another exemplary embodiment, the plate can include features to facilitate mating with a guide device. Exemplary features include, for example, a notch and/or a mating edge formed on a perimeter of the plate. In certain exemplary embodiments, the plate includes a notch formed in the proximal and/or distal end of the elongate central portion and at least one mating edge formed on one or both branch portions. The mating edge(s) can extend substantially perpendicular to the longitudinal axis of the central portion and it can be opposed to the notch so that a guide device can engage the mating edge and the notch.

In another embodiment, a guide device is provided having a guide member with at least one pathway formed therethrough for receiving various tools, devices, and implants, such as bone preparation tools (e.g., awls, drill bits, taps, flexible shaft drills, universal joint taps, etc.), driver devices (screwdriver, universal joint screwdrivers, flexible shaft screwdrivers, etc.), and fasteners (e.g., bone screws, etc.). The guide device can also include features to mate the guide device to a spinal plate and to align the pathway(s) in the guide device with one or more thru-bore(s) in the plate. For example, the guide device can have at least one protrusion that is adapted to engage a notch in a spinal plate, and/or at least one deflectable member that is adapted to engage an edge formed on the spinal plate. In an exemplary embodiment, the protrusion(s) extends distally from opposed ends of the distal end of the guide member at a location that is substantially adjacent to the outer perimeter of the distal end of the guide member, and the deflectable member(s) extends distally from a substantial mid-portion of the guide member.

In other embodiments, a spinal kit is provided having a spinal plate and a guide device that is adapted to engage the plate to align at least one pathway in the guide device with at least one thru-bore formed in the spinal plate. The plate and/or guide device can include features to facilitate mating with one another. Exemplary features include one or more protrusions on the guide device that are adapted to engage one or more notches on the plate, and/or one or more deflectable members on the guide device that are adapted to engage one or more edges of the spinal plate. In another exemplary embodiment, the guide device can mate to the plate in a first orientation and in a second orientation different than the first orientation. The first and second orientations can be opposite to one another, such that the guide device is reversibly matable to the plate.

Also disclosed herein are methods for implanting a spinal fixation plate using a guide device.

### BRIEF DESCRIPTION OF THE DRAWINGS

FIG. 1A is a top perspective view of an exemplary embodiment of a spinal fixation plate;

FIG. 1B is a bottom view of the spinal fixation plate shown in FIG. 1A;

FIG. 2 is a top perspective view of the spinal fixation plate shown in FIG. 1A with anchoring elements mated thereto;

FIG. 3A is a side perspective view of an exemplary embodiment of a guide device;

FIG. 3B is a side perspective view of the guide member of the guide device shown in FIG. 3A;

FIG. 3C is a bottom view of the guide member shown in FIG. 3B;

FIG. 3D is a side view of the guide member shown in FIG. 3B;

FIG. 4A is a top perspective view of the guide device shown in FIG. 3A mated to the spinal fixation plate shown in FIG. 1A;

FIG. 4B is a bottom perspective view of the guide device and spinal fixation plate shown in FIG. 4A;

FIG. 5A is a side perspective view of a straight drill for use with a spinal fixation kit;

FIG. 5B is a side perspective view of a flexible drill for use with a spinal fixation kit;

FIG. 6 is a drill stop for use with a exemplary spinal fixation kit;

FIG. 7A is a straight tap for use with an exemplary spinal fixation kit;

FIG. 7B is a universal tap for use with an exemplary spinal fixation kit; and

FIG. 8 is a screw driver for use with an exemplary spinal fixation kit.

### DETAILED DESCRIPTION OF THE EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Disclosed herein are an implantable spinal fixation plate and a guide device for implanting the spinal fixation plate. In an exemplary embodiment, the guide device and the spinal fixation plate can include features that allow the two devices to removably mate to one another. As a result, the guide device can be used to position and hold the plate against bone while inserting drills, taps, awls, and other bone preparation devices through the guide device. The guide device can also be configured to allow bone screws or other implants to be inserted therethrough to attach the plate to bone.

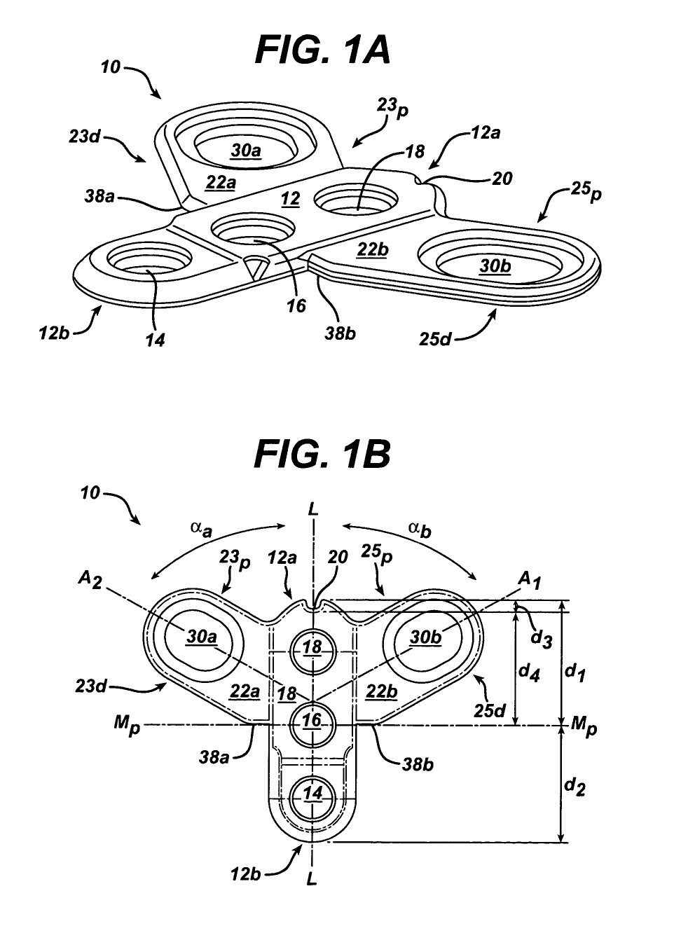

FIGS. 1A-1B illustrate one exemplary embodiment of a spinal fixation plate 10 that is adapted to be implanted in the occiput of a patient's spine. As shown, the plate 10 has a generally elongate central portion 12 that defines a longitudinal axis L extending between proximal and distal ends 12a, 12b thereof, and first and second branch portions 22a, 22b that extend from opposed sides of the central portion 12. The elongate central portion 12 can be used to attach the plate 10 to the occiput, and the branch portions 22a, 22b can be configured to mate a spinal fixation element, such as, by way of non-limiting example, a spinal fixation rod, cable, tether, or another spinal plate, to the plate 10. While plate 10 is an occipital plate, a person skilled in the art will appreciate that the plate 10 can have a variety of other configurations and that the various features disclosed herein can be adapted for use on virtually a spinal plate having virtually any configuration, for instance, a T configuration, an M configuration, a Y configuration, or a cross configuration.

The shape of the elongate central portion 12 can vary, but in an exemplary embodiment the elongate central portion 12 includes proximal and distal ends 12a, 12b having a rounded or convex profile to avoid the risk of damage during implantation. The length of the elongate central portion 12 can also vary, and the length will depend on the number of thru-bores formed therein. While the elongate central portion 12 can include any number of thru-bores formed therein for receiving a fastening element for mating the plate 10 to bone, FIGS. 1A-1B illustrate three thru-bores 14, 16, 18 formed within the elongate central portion 12. In particular, the central portion 12 includes a first proximal thru-bore 14, a second central or middle thru-bore 16, and a third distal thru-bore 18. The thru-bores 14, 16, 18 can be aligned with one another along the longitudinal axis L of the elongate central portion 12, and each thru-bore 14, 16, 18 can be equidistant from one another. A person skilled in the art will appreciate that the plate 10 can include any number of thru-bores formed therein, and that the thru-bores can be positioned anywhere on the central portion 12 or elsewhere on the plate 10 depending upon the intended use. The thru-bores 14, 16, 18 can also vary in shape and size depending upon the intended use of the plate 10 and the function of each thru-bore 14, 16, 18. In the illustrated embodiment, each thru-bore 14, 16, 18 has a substantially circular shape.

The first and second branch portions 22a, 22b that extend from opposed sides of the central portion 12 can also have a variety of configurations. In the illustrated embodiment, each branch portion 22a, 22b has a generally elongate shape with proximal and distal edges 23p, 23d, 25p, 25d and a substantially rounded terminal end 27a, 27b. Each branch portion 22a, 22b is positioned just distal to the proximal end 12a of the elongate central portion 12, such that the proximal end 12a of the elongate central portion 12 extends proximally beyond the location at which the branch portions 22a, 22b are attached to the elongate central portion 12. Each branch portion 22a, 22b can also extend at an angle αₐ, α_{b} relative to the longitudinal axis L of the elongate central portion 12. While the angle αₐ, α_{b} can vary depending on the intended use, in the illustrated embodiment each branch portion 22a, 22b extends along a central axis A₁, A₂ that is disposed at an acute angle αₐ, α_{b} relative to the longitudinal axis L of the elongate central portion 12, as measured toward the proximal end 12a of the elongate central portion 12.

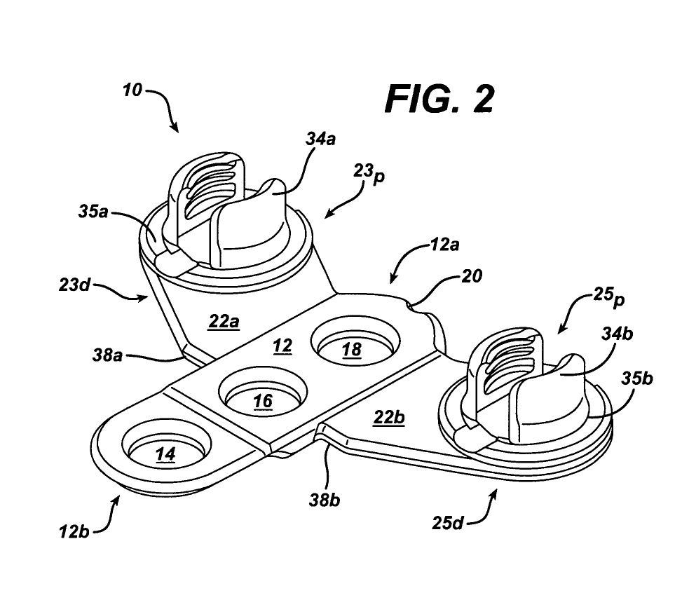

The branch portions can also include at least one thru-bore or slot 30a, 30b formed therein for attaching a spinal fixation element, such as, by way of non-limiting example, a spinal rod, tether, cable, or another plate, to the plate 10. The shape of each thru-bore 30a, 30b formed in each branch portion 22a, 22b can vary depending on the intended use. By way of non-limiting example, each thru-bore 30a, 30b can have an oblong or ovular shape, as shown in FIGS. 1A-1B, or they can have a circular shape or any other shape. An oblong or ovular shape is advantageous in that it allows an anchoring assembly to be mated to the plate 10 and adjusted as desired relative to the branch portion 22a, 22b. Anchoring assemblies are known in the art, and they are typically used to attach a spinal fixation element, such as a spinal rod, to a spinal fixation plate. FIG. 2 illustrates one exemplary embodiment of an anchoring assembly mated to the thru-bore 30a, 30b in each branch portion 22a, 22b. In general, each anchoring assembly includes a rod-receiving member 34a, 34b that extends through the thru-bores 30a, 30b in the plate 10, and a fastening element 35a, 35b, in the form of a snap ring that is adapted to engage the rod-receiving member 34a, 34b to mate the rod-receiving member 34a, 34b to the spinal fixation plate 10. A person skilled in the art will appreciate that a variety of anchoring assemblies and other techniques can be used to mate a spinal fixation element, such as a spinal rod, to the spinal plate 10, and that the anchoring assembly or other mating device can be fixedly attached to or integrally formed with the spinal fixation plate 10. Moreover, as previously indicated above, the various exemplary features disclosed herein can be incorporated into virtually any spinal plate, and thus the spinal plate does not need to include branch portions, much less any type of anchoring assembly for mating the plate to a spinal fixation element.

In another exemplary embodiment, the spinal fixation plate 10 can include features to facilitate mating of the plate 10 with a guide device, which will be discussed in more detail below. While various mating features and techniques can be used, in certain exemplary embodiments the plate 10 can include at least one notch and/or at least one mating edge. As shown in FIGS. 1A-1B, the exemplary plate 10 includes a notch 20 formed in the proximal end 12a of the central portion 12 and a mating edge 38a, 38b formed on each branch portion 22a, 22b. The notch 20 can be formed at a variety of locations on the plate 10, but in an exemplary embodiment it is adapted to receive a protrusion on a guide device such that the guide device is prevented from rotating relative to the plate 10 when mated thereto. The notch 20 can also be adapted to function as a centering mechanism to center the thru-bores 14, 16, 18 in the plate 10 with the one or more pathways in a guide device. As shown in FIG. 1B, the notch 20 is formed on the perimeter of the proximal end 12a of the elongate central portion 12 and it is aligned with the longitudinal axis L thereof. The shape of the notch 20 can also vary, but in an exemplary embodiment it has a shape that complements the shape of a corresponding protrusion on a guide device. As shown in FIGS. 1A-1B, the notch 20 has a semi-circular shape for receiving a substantially cylindrical protrusion on a guide device. One skilled in the art will appreciate that the notch 20 can be formed at a variety of other locations on the plate 10, and it can have virtually any shape and size, such as triangular, rectangular, or square shaped. Moreover, the plate 10 does not necessarily need to include a notch 20, but rather it can have other features, such as a bore or protrusion, to facilitate alignment of a guide device with the plate 10.

As indicated above, in certain exemplary embodiments the plate 10 can include one or more mating edges, such as mating edges 38a and 38b formed on the branch portions 22a, 22b. The mating edges 38a, 38b can have a variety of configurations and they can be formed anywhere on the branch portion 22a, 22b, or elsewhere on the plate 10. In the illustrated exemplary embodiment, the mating edges 38a, 38b are generally planar edges that are formed on the distal edge 23d, 25d of each branch portion 22a, 22b. In particular, each mating edge 38a, 38b can extend perpendicular to the longitudinal axis L of the elongate central portion 12. As a result, the mating edges 38a, 38b, can extend at an angle relative to the distal edge of the branch portions 22a, 22b. The length of the mating edges 38a, 38b can also vary, but in an exemplary embodiment they have a length that is sufficient to receive a corresponding deflectable member on a guide device, as will be discussed in more detail below.

In another exemplary embodiment, the mating edges 38a, 38b on the plate 10 can function in combination with the notch 20 to allow a guide device to reversibly mate to the plate 10 in two orientations. This will be discussed in more detail below.

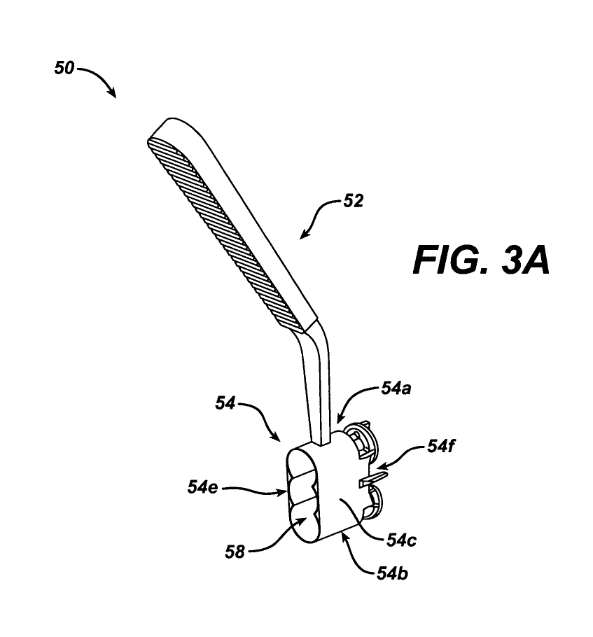

As previously indicated, a guide device for use in implanting a spinal fixation plate is also provided. While the guide device can have a variety of configurations and it can be adapted for use with a variety of fixation plates, FIGS. 3A-3D illustrate one exemplary embodiment of a guide device 50 for use with the spinal fixation plate 10 shown in FIGS. 1A-1B. As shown, the guide device 50 includes a guide member 54 having a substantially rectangular, elongate shape with a handle 52 attached thereto. For reference purposes, the guide member 54 will be referred to as having opposed first and second lateral sidewalls 54c, 54d connected by opposed end walls 54a, 54b. The lateral sidewalls 54c, 54d and end walls 54a, 54b extend between opposed proximal and distal ends 54e, 54f of the guide member 54. As indicated above, the guide device 50 may include a handle 52 to facilitate grasping and manipulation of the device 50. The handle 52 can be attached to the guide member 54 at a variety of locations, but in the illustrated exemplary embodiment shown in FIG. 3A the handle 52 extends from the end wall 54a of the guide member 54. The handle 52 can also have a variety of configurations, but in the illustrated exemplary embodiment the handle 52 is in the form of a substantially cylindrical shaft having a couple of bends formed therein which allow the handle 52 to be offset from the guide member 54. The handle 52 can also include features to facilitate gripping, such as a knurled surface, ridges, or grooves. In another embodiment, while not shown, the handle 52 or the guide member 54 can include a clamp member formed thereon or mated thereto that is effective to mate the guide device 50 to a surgical retractor, or to a support. A person skilled in the art will appreciate that a variety of clamp members and/or other mating techniques can be used to mate the guide device 50 to a retractor or other type of support member.

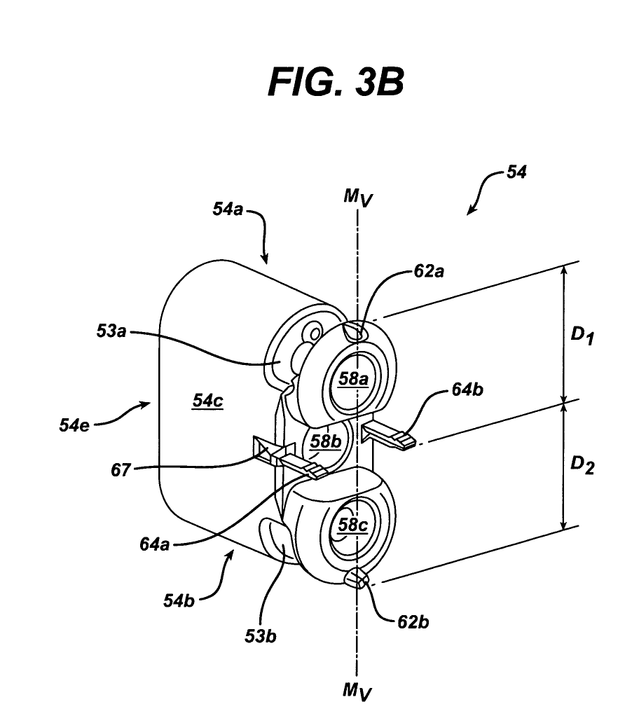

The guide member 54 can also have a variety of configurations, but in one exemplary embodiment it includes at least one pathway 58 formed therethrough for receiving various tools, devices, and implants, such as bone preparation tools (e.g., awls, drill bits, taps, flexible shaft drills, universal joint taps, etc.), driver devices (screwdrivers, universal joint screwdrivers, flexible shaft screwdrivers, etc.), and fasteners (e.g., bone screws, etc.). In an exemplary embodiment, the pathway 58 extends between the proximal and distal ends 54e, 54f of guide member 54, and it is adapted to be aligned with one or more corresponding thru-bores formed in a spinal fixation plate to provide a fixed entry angle for a tool, device, or implant being inserted therethrough. In the embodiment shown in FIGS. 3A-3D, the pathway 58 in the guide member 54 includes a distal portion 58d with three separate lumens 58a, 58b, 58c that extend to an open proximal portion 58p. The open proximal portion 58p of the pathway 58 allows an enlarged diameter portion of a tool to be received therein, while a reduced diameter tip of the tool extends through one of the distal lumens 58a, 58b, 58c in the guide member 54. By way of non-limiting example, FIG. 4A illustrates a drill bit 80 having a depth-stop sleeve 82 disposed therearound for limiting penetration of the tip 84 of the drill bit 80 into bone. The sleeve 82 is received within the open proximal portion 58p of the pathway 58, while the tip 84 extends through distal lumen 58b in the guide member 54. As is further shown in FIG. 4A, the open proximal portion 58p of the pathway 58 can also define distinct regions for guiding a tool toward one of the distal lumens 58a, 58b, 58c. For example, in the illustrated embodiment, the open proximal portion 58p optionally includes three substantially cylindrical regions 59a, 59b, 59c that are co-axial with the three distal lumens 58a, 58b, 58c, and that are in either partial or entire communication with one another. The cylindrical regions 59a, 59b, 59c are defined by the inner sidewalls of the guide member 54.

A person skilled in the art will appreciate that the guide member 54 can include any number of pathways or lumens extending therethrough, and that each pathway or lumen can have a variety of other configurations. By way of non-limiting example, the guide member 54 can include only lumens, e.g., one, two, etc., formed therein and adapted to be aligned with corresponding thru-bores formed in a spinal plate, and the lumens can be separate from one another or they can be partially or entirely in communication with one another.

The guide member 54 can also include one or more cut-out portions or windows 53a, 53b formed therein to facilitate visual access to a spinal fixation plate coupled to the guide device 50. The cut-out portions 53a, 53b can be formed anywhere in the guide member 54, such as, for example, in one or more of the end and/or lateral sidewalls 54a, 54b, 54c, 54d of the guide member 54. In an exemplary embodiment, as shown in FIGS. 3B and 3D, each end sidewall 54a, 54b includes a cut-out portion 53a, 53b formed therein for providing visual access to the pathway 58 and to tools, devices, and implants being inserted therethrough.

The guide member 54 can also include one or more mating features to facilitate mating of the guide device 50 with a spinal fixation plate, such as plate 10 shown in FIGS. 1A-1B. In the illustrated exemplary embodiment, the guide member 54 includes first and second protrusions 62a, 62b formed on opposed ends of the distal end 54f, and first and second deflectable members or tabs 64a, 64b formed at a substantial mid-portion of the distal end 54f of the guide member 54. The protrusions 62a, 62b and deflectable members 64a, 64b can be configured to engage the notch 20 and mating edges 38a, 38b of plate 10 to align each lumen 58a, 58b, 58c in the guide member 54 with the thru-bores 14, 16, 18 in the plate 10.

The protrusions 62a, 62b can have a variety of configurations and they can be positioned anywhere on the distal end 54f of the guide member 54. In an exemplary embodiment, at least one of the protrusions 62a, 62b is adapted to engage the notch 20 formed in the spinal fixation plate 10, while the other protrusion 62a, 62b rests against or abuts the opposed end, e.g., the distal end 12b of the plate 10. Accordingly, as shown in FIGS. 3B-3D, the protrusions 62a, 62b extend distally from the distal end 54f of the guide member 54 substantially adjacent to the opposed end walls 54a, 54b of the guide member 54. The protrusions 62a, 62b can also have a variety of shapes, but in an exemplary embodiment each protrusion 62a, 62b has a shape that allows the protrusion 62a, 62b to be received within the notch 20. As shown in FIGS. 3B-3D, each protrusion 62a, 62b has a cylindrical shape. Other embodiments may have only one protrusion.

Each deflectable member 64a, 64b can also have a variety of configurations, but in an exemplary embodiment they are adapted to abut and/or engage the mating edge(s) 38a, 38b of the plate 10. As shown in FIG. 3B, the deflectable members 64a, 64b have a substantially planar configuration and they extend distally from opposed sides of a mid-line Mₕ of the distal end 54f of the guide member 54. More particularly, the deflectable members 64a, 64b are formed within and extend from a recess 67 formed in the distal end 54f of the guide member 54. As a result of the recess 67, the deflectable members 64a, 64b can have a length that is sufficient to allow the members 64a, 64b to deflect when they engage a spinal plate. A person skilled in the art will appreciate that the deflectable members 64a, 64b can have a variety of other configurations, and that various other techniques can be used to engage a spinal plate with the guide member 54. For example, the mating edges 38a, 38b of the plate 10 can be made deflectable by adding a relief cut into the plate 10, and the deflectable members 64a, 64b of the guide member 54 can be rigid. Moreover, the deflectable member(s) 64a, 64b can be formed at a variety of other locations on the guide member 54.

As previously indicated, in certain exemplary embodiments the guide device 50 can be configured to reversibly engage the spinal fixation plate 10. In particular, the guide device 50 can be engage the plate 10 in a first orientation in which lumen 58a is aligned with thru-bore 14, lumen 58b is aligned with thru-bore 16, and lumen 58c is aligned with thru-bore 18, and in a second, opposite orientation in which lumen 58a is aligned with thru-bore 18, lumen 58b is aligned with thru-bore 16, and lumen 58c is aligned with thru-bore 14. While this reversible orientation can be achieved using a variety of techniques, in one exemplary embodiment, the protrusions 62a, 62b on the guide member 50 are equidistant from the deflectable members 64a, 64b, and the plate 10 has a configuration that allows the guide member 54 to engage the plate 10 in both orientations. Referring back to FIG. 1B, the distance d₁ between the proximal end 12a and the midline Mₚ of the plate 10 can be greater than the distance d₂ between the distal end 12b and the midline Mₚ of the plate 10 to compensate for the depth d₃ of the notch 20, such that either protrusion 62a, 62b on the guide member 50 can be positioned within the notch 20 while the deflectable members 64a, 64b abut against the mating edges 38a, 38b. As a result of the difference between d₁ and d₂, the mating edges 38a, 38b on the plate 10 can therefore be offset from the mid-line Mₚ of the elongate central portion 12. In an exemplary embodiment, the mating edges 38a, 38b are toward the proximal end 12a of the plate 10 by a difference that is slightly less than a depth d₃ of the notch 20 to cause the deflectable members 64a, 64b to deflect against the mating edges 38a, 38b and thereby engage the plate 10 by an interference fit. This also results in d₄, which is equal to d₁ minus d₃, being greater than d₂.

A person skilled in the art will appreciate that the protrusions 62a, 62b and/or deflectable members 64a, 64b can vary depending upon the configuration of the spinal plate 10 and the corresponding mating features on the plate 10.

In use, as shown in FIGS. 4A-4B, the pathway 58, and in particular each lumen 58a, 58b, 58c, in the guide member 54 can be aligned with the thru-bores 14, 16, 18 in the elongate central portion 12 of the plate 10. This can be achieved by juxtapositioning the guide member 54 on the plate 10, as shown in FIGS. 4A and 4B, such that one of the protrusion(s) 62a, 62b on the guide member 54, e.g., protrusion 62a, engages the notch 20 on the plate 10, and the other protrusions 62a, 62b, e.g., protrusion 62b rests against or is clear of the distal end 12b of the plate 10. As a result, the deflectable members 64a, 64b on the guide member 54 can abut against and deflect relative to the mating edges 38a, 38b on the plate 10, thereby creating an interference fit between protrusion 62a and the deflectable members 64a, 64b to engage the plate 10. As indicated above, depending upon the particular configuration of the plate 10 and guide device 50, the guide device 50 can reversibly mate to the plate 10. This allows the handle 52 (as shown in FIG. 3A) on the guide device 50 to be positioned on either side of the plate 10, as so may be desired. One skilled in the art will appreciate that when the spinal plate is an occipital plate the ability to allow the handle to be positioned on either side of the plate is important because the plate can be placed on the occiput in more than one position.

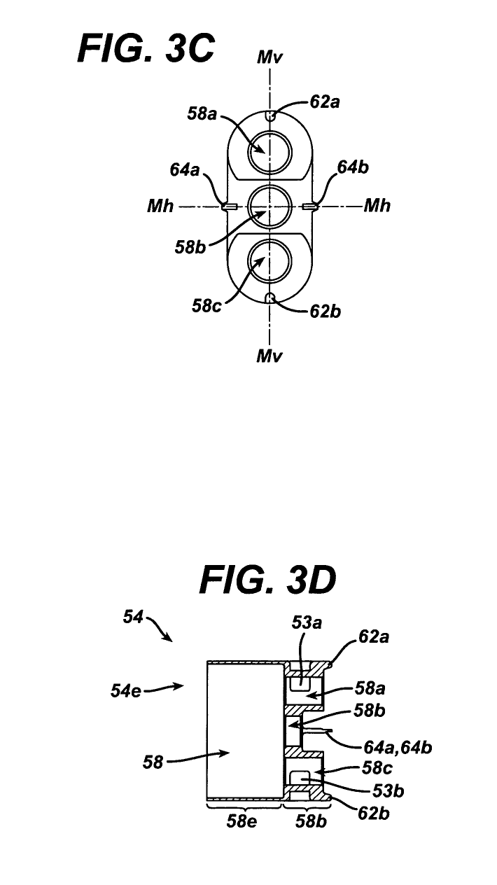

Once the guide device 50 and the plate 10 are mated to one another, the plate 10 can be placed against the occiput. The bone can then be prepared to attach the plate 10 to the bone. In particular, bone preparation tools, such as drills, taps, awls, etc., can be passed through one or more of the lumens 58a, 58b, 58c in the pathway 58 in the guide member 54 to form a bone hole in bone at one of more of the thru-bores 14, 16, 18 in the plate 10. For example, as previously described, FIGS. 4A and 4B illustrate a drill bit 80 disposed through lumen 58b in the pathway 58 for forming a bone hole in bone under the middle thru-bore 16 in the plate 10. FIGS. 5A-5B also illustrate exemplary embodiments of drills 70, 70′ which can be disposed one or more of the lumens 58a, 58b, 58c in the pathway 58 for forming a bone hole in bone. In the embodiment shown in FIG. 5A, the drill 70 includes a shaft 74 having a proximal end 74a that is adapted to mate to a driver mechanism and a distal end 74b in the form of a drill bit for drilling a hole in bone. Similarly, in the embodiment shown in FIG. 5B, the drill 70′ includes a shaft 74′ having a proximal end 74a′ that is adapted to mate to a driver mechanism, and a distal end 74b′ in the form of a drill bit for preparing a hole in bone. Drill 70′, however, includes a flexible portion 75 extending between the proximal and distal ends 74a′, 74b′ that allows the distal end 74b′ to be positioned at various angles relative to the proximal end 74a′. While the flexible portion 75′ can have a variety of configurations, in the illustrated embodiment the flexible portion 75′ is formed from two coils that are wound in opposite directions.

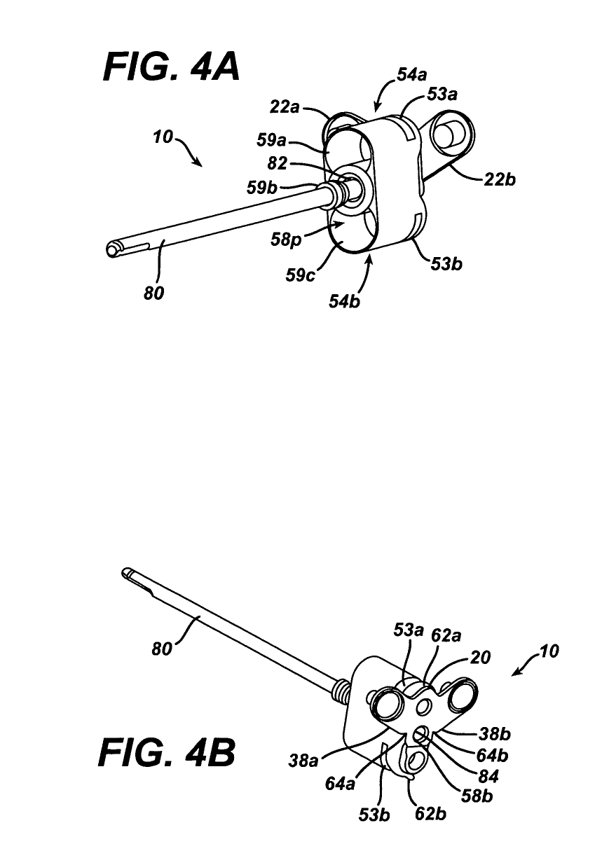

The drills 70, 70′ can also optionally be used in combination with a drill stop 80, which is shown in FIG. 6. The drill stop 80 is adapted to limit the penetration depth of the drill into bone. As shown, the drill stop 80 has a generally cylindrical shape with an opening 82 extending and therethrough. The drill stop 80 is adapted to be disposed over a portion of the shaft of a drill. For example, the drill stop 80 can be disposed over and engage notches 77, 77′ formed on a portion 78, 78′ of the shaft that is disposed just proximal to the distal end 74b, 74b′ on drills 70 and 70′. In use, the drill stop 80 can be received within the cylindrical regions 59a, 59b, 59c of the guide member 54 to limit penetration of the drill 70, 70′ through the lumen 58a, 58b, 58c and into bone.

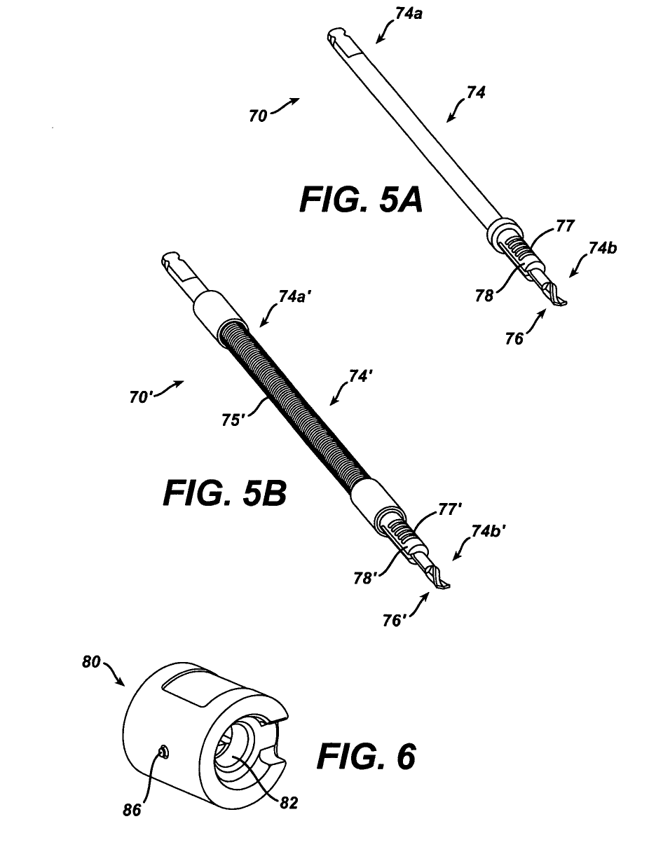

Once the bone hole(s) are prepared, a tap can be used to form threads within the bone hole(s). By way of non-limiting example, FIGS. 7A-7B illustrate taps 90, 90′ which can be disposed through one or more of the lumens 58a, 58b, 58c in the pathway 58 for forming threads in the bone hole(s). As shown, each tap 90, 90′ generally includes a shaft 94, 94′ with a proximal end 94a, 94a′ and a distal end 94b, 94b′. The proximal end 94a, 94a′ of each tap 90, 90′ can fixedly or integrally mate to a handle (not shown) and the distal end 94b, 94b′ of each tap 90, 90′ includes threads formed thereon for forming threads in a bone hole. The shaft 94, 94′ can have a rigid, substantially straight configuration, as shown in FIG. 7A, or it can be configured to allow the distal end 94b′ to be angularly adjustable relative to the proximal end 94a′. While the angular movement of the shaft 94, 94′ can be achieved in a variety of ways, FIG. 7B illustrates a U-shaped connector or Universal joint 95′ formed on the shaft for allowing the distal portion of the shaft 94′ to pivot relative to the proximal portion of the shaft 94′. Other embodiments may have a knuckle joint, cardan joint, or a fixed angled configuration driven by gears.

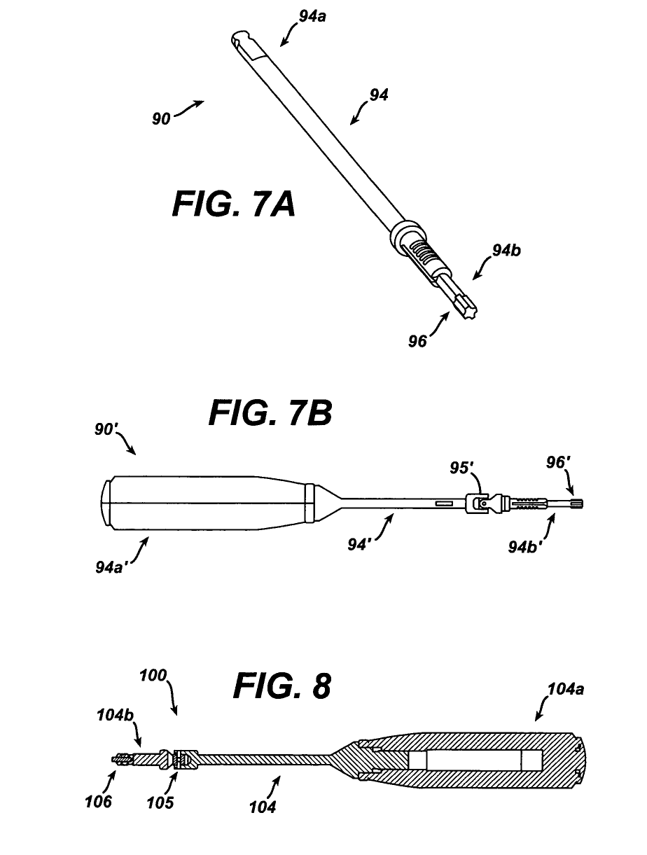

Once the bone hole(s) are tapped, one or more fastening elements, such as bone screws, can be passed through the guide device to attach the plate 10 to bone. By way of non-limiting example, FIG. 8 illustrates one exemplary embodiment of a screw driver 100 which can be disposed through one or more of the lumens 58a, 58b, 58c in the guide member 54 for inserting a screw in one or more of the bone holes. While the screw driver 100 can have a variety of configurations, as shown the screw driver 100 has a shaft 104 having a proximal end 104a that can be configured to fixedly or integrally mate with a handle or a drive mechanism, and a distal end 104b that is adapted to engage and retain a fastening element, such as a bone screw, to drive the fastening element into bone. As was previously described with respect to tap 90′ shown in FIG. 7B, the screw driver 100 can be adapted to allow the distal end 104b to be positioned at various angles relative to the proximal end 104a. While angular movement of the shaft 104 can be achieved in a variety of ways, FIG. 8 illustrates a U-shaped or Universal Joint connector 105, similar to that described above, incorporated into the shaft 104.

Once the plate 10 is attached to bone, in an exemplary embodiment a spinal fixation element, such as a spinal rod, cable, tether, or another plate, can be attached to the plate 10, and in particular to the branch portions 22a, 22b using the anchoring elements 34a, 34b. The exemplary anchoring element will be locked with an inner set screw. One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A spinal fixation kit, comprising:
a substantially planar implantable spinal fixation plate having proximal and distal ends that define a longitudinal axis extending therebetween and at least one thru-bore formed therein;
a guide device having proximal and distal ends with a pathway extending therethrough, the distal end of the guide device including at least one protrusion and at least one deflectable member that is adapted to engage a perimeter of the spinal plate by interference fit to align the at least one pathway in the guide device with the at least one thru-bore formed in the spinal fixation plate.

2. The spinal fixation kit of claim 1, wherein the spinal fixation plate includes at least one mating edge and at least one notch formed on a perimeter thereof, and wherein the at least one protrusion on the guide device is adapted to be received within the at least one notch on the spinal fixation plate and the at least one deflectable member on the guide device is adapted to engage the at least one mating edge formed on the spinal fixation plate.

3. The spinal fixation kit of claim 2, wherein the at least one pathway in the guide member comprises three pathways, and the at least one thru-bore in the central portion of the plate comprises three thru-bores, and wherein the at least one protrusion and the at least one deflectable on the guide member are adapted to engage the at least one notch and the at least one mating edge on the spinal fixation plate such that the three pathways in the guide member can be aligned with the three thru-bores in the plate in a first direction and a second opposite direction.

4. A spinal fixation kit, comprising:
a spinal fixation plate having a plurality of thru-bores formed therein; and
a guide member having a plurality of pathways extending therethrough and at least one mating feature adapted to engage the spinal fixation plate to align the plurality of pathways in the guide member with the plurality of thru-bores in the spinal fixation plate, the at least one mating feature being configured to engage the spinal fixation plate in a first orientation and in a second orientation that is opposite to the first orientation.

5. The spinal fixation kit of claim 4, wherein the at least one mating element comprises at least one protrusion extending from a distal end of the guide member and adapted to engage a corresponding notch formed on the spinal fixation plate, and at least one deflectable member extending distally from the distal end of the guide member and adapted to engage a mating edge formed on the spinal fixation plate.

6. The spinal fixation kit of claim 5, wherein the at least one protrusion and the at least one deflectable member are adapted to engage the plate by an interference fit.

7. The spinal fixation kit of claim 4, wherein the guide member includes first and second protrusions formed on opposed ends of the distal end thereof, and first and second deflectable member opposed to one another and formed at a substantial mid-portion of the distal end of the guide member.

8. The spinal fixation kit of claim 7, wherein the spinal fixation plate includes an elongate central portion having a longitudinal axis extending between proximal and distal ends, and first and second branch portions that extend transverse to the longitudinal axis from opposed sides of the elongate central portion.

9. The spinal fixation kit of claim 8, wherein each branch portion includes a mating edge formed on a distal edge thereof, each mating edge extending substantially perpendicular to a longitudinal axis of the central portion, and wherein the notch is formed in the proximal end of the spinal fixation plate.

10. The spinal fixation kit of claim 4, wherein the at least one pathway in the guide member comprises three lumens extending therethrough, and wherein the kit further includes at least one bone screw that is adapted to extend through a lumen in the guide member and through a thru-bore in the plate to attach the plate to bone.

11. A spinal fixation kit, comprising:
a guide device having at least one pathway extending therethrough between proximal and distal ends thereof, the guide device being adapted to engage a spinal fixation plate to position the spinal fixation plate adjacent to bone and to align the at least one pathway extending through the guide device with at least one thru-bore formed in the spinal fixation plate; and
at least one instrument adapted to be inserted through a pathway in the guide device, the instrument having a distal end that is adapted to be positioned at an angle relative to a proximal end of the instrument.

12. The spinal fixation kit of claim 11, wherein the distal end of the instrument is positioned at a fixed angle relative to the proximal end of the instrument.

13. The spinal fixation kit of claim 11, wherein the distal end of the instrument is angularly adjustable relative to the proximal end of the instrument.

14. The spinal fixation kit of claim 11, wherein the instrument comprises a drill having a flexible shaft extending between the proximal and distal ends.

15. The spinal fixation kit of claim 11, wherein the instrument includes an elongate shaft extending between the proximal and distal ends and having a connector formed thereon and adapted to allow the distal end to pivot relative to the proximal end.

16. The spinal fixation kit of claim 15, wherein the instrument comprises a tap.

17. The spinal fixation kit of claim 15, wherein the instrument comprises a screw driver.

18. The spinal fixation kit of claim 11, further comprising a spinal fixation plate having an elongate central portion with at least one thru-bore formed therein and adapted to be aligned with the at least one pathway extending through the guide device, and first and second branch portions extending from opposed sides of the elongate central portion.

19. A spinal fixation kit, comprising:
a guide device having at least one pathway extending therethrough between proximal and distal ends thereof;
a spinal fixation plate having at least one thru-bore formed therein and adapted to be aligned with the at least one pathway extending through the guide device; and
a bone fastener adapted to be disposed through a pathway in the guide device and through a thru-bore formed in the spinal fixation plate to attach the spinal fixation plate to bone.

20. The spinal fixation kit of claim 19, wherein the bone fastener comprises a bone screw.
